(19) United States
(12) Patent Application Publication
(10) Pub. No.: US 2023/0181059 A1
(43) Pub. Date: Jun. 15, 2023

(54) SYSTEMS AND METHODS FOR ONGOING MONITORING OF HEALTH AND WELLBEING

(71) Applicant: VAYYAR IMAGING LTD., Yehud (IL)

(72) Inventors: Michael ORLOVSKY, Hod Ha-Sharon (IL); Ronen TUR, Binyamina (IL); Yuval LOMNITZ, Herzeliya (IL); Tsachi ROSENHOUSE, Kiryat Ono (IL); Amit DVASH, Kadima-Zoran (IL); Ofer FAMILIER, Tel Aviv (IL); Shay MOSHE, Petach-Tikva (IL); Rotem BARDA, Tel Aviv (IL); Tal DRUYAN KATZ, Chicago, IL (US); Adi PAZ, Herzeliya (IL); Eyal KOREN, Rehovot (IL)

(21) Appl. No.: 17/924,998

(22) PCT Filed: May 14, 2021
(86) PCT No.: PCT/IB2021/054130

§ 371 Date: Nov. 13, 2022

Related U.S. Application Data
(60) Provisional application No. 63/024,520, filed on May 14, 2020.
(60) Provisional application No. 63/042,023, filed on Jun. 22, 2020.
(60) Provisional application No. 63/093,319, filed on Oct. 19, 2020.

(51) Int. Cl. A61B 5/11; A61B 5/00

(52) U.S. Cl. CPC A61B 5/112; A61B 5/1113; A61B 5/7264

(57) ABSTRACT

Systems and methods for monitoring health and wellbeing in an ongoing manner use radar chips to scan monitored regions such that data obtained by the scanning radar chip are processed to identify targets within the monitored region. Health indication and activity parameters are gathered via a wellbeing prediction engine assesses the targets using a machine learning model.

### CROSS-REFERENCE TO RELATED APPLICATIONS

This application claims the benefit of priority from U.S. Provisional Patent Application No. 63/024,520, filed May 14, 2020, U.S. Provisional Patent Application No. 63/042,037, filed Jun. 22, 2020 and U.S. Provisional Patent Application No. 63/093,319, filed Oct. 19, 2020, the contents of which are incorporated by reference in their entirety.

### FIELD OF THE INVENTION

The disclosure herein relates to systems and methods for monitoring health and wellbeing in an ongoing manner. In particular the disclosure relates to radar based a subject monitoring system operable to collect risk parameters in communication with a prediction engine.

### BACKGROUND

Ongoing assessment of health and wellbeing, particularly in the home, may be an effective way to identify early indications of the onset of ailments such that early medical intervention may be offered thereby reducing and in many cases avoiding altogether the need for hospitalization.

For example, ongoing monitoring of various health parameters indicative of health may be allow a reasonable estimation of a subjects risk of developing chronic heart failure (CHF) or the like. Thus typically subjects are required to actively collect health parameters. Typically, subjects may be required to regularly weigh themselves and to report their weight to health practitioners. Regular data may indicate sudden weight increase which is characteristic of the onset of CHF.

Nevertheless, subjects do not generally monitor or report their health parameters with sufficient regularity for such predictions to be useful. As a result, the onset of CHF often goes undetected and preventative measures are not timely taken to prevent deterioration.

The need remains, therefore, for more efficient systems and methods monitoring health in an ongoing manner and assessing risk of ailments such as chronic heart failure (CHF). The invention described herein addresses the above-described needs.

### SUMMARY OF THE EMBODIMENTS

According to one aspect of the presently disclosed subject matter, a system is introduced for monitoring the ongoing wellbeing of at least one subject comprising: at least one subject monitoring station configured and operable to collect health indication parameters from the at least one subject. The subject monitoring station may comprise: at least one radar unit comprising at least one transmitter antenna connected to an oscillator and configured to transmit electromagnetic waves towards a target region, and at least one receiver antenna configured to receive electromagnetic waves reflected by a subject located within the target region and operable to generate raw data; and at least one processor configured to receive raw data from the radar unit and operable to generate said health indication parameters.

An activity monitor may be configured and operable to record events indicative of activity of daily living of the at least one subject. A memory unit may be configured to store recorded data generated by the subject monitoring station and the activity monitor. At least one wellbeing prediction engine may comprise a processor configured and operable to access the recorded data stored in the memory unit and to execute a wellbeing predictive function thereby generating at least one wellbeing index for the at least one subject. Additionally or alternatively, a communication module may be configured and operable to communicate information to third parties.

Variously, the subject monitoring station may comprises a body volume monitor configured and operable to calculate a body volume index of the at least one subject, a remote vital signs monitor operable to record breathing rate and heart rate of the subject, at least one heart rate monitor operable to record heart rate of the subject, at least one breathing rate monitor operable to record breathing rate of the subject, at least one body temperature monitor operable to record body temperature of the subject, at least one blood pressure monitor operable to record blood pressure of the subject, at least one body weight monitor operable to record body weight of the subject, a gait speed monitor or the like as well as combinations thereon.

Optionally, a gait speed monitor may include a processor comprising: a data filter configured to receive said raw data, and operable to process the raw data to remove data relating to reflections from static objects thereby generating filtered data; a tracker module configured to receive the filtered data from the data filter and operable to process the filtered data to identify moving targets and to track the location of the moving targets over time thereby generating target data; and a gait classification module configured to receive the target data from the tracker module and operable to process the target data by applying gait classification rules and further operable to calculate a gait speed of the subject.

Where appropriate, at least one wellbeing prediction engine may comprise a Chronic Heart Failure (CHF) prediction engine comprising a processor configured and operable to access the recorded data stored in the memory unit and to execute a Chronic Heart Failure (CHF) predictive function thereby generating a CHF risk index for the subject. The Chronic Heart Failure (CHF) predictive function may receive input parameters selected from a group consisting of: activity of daily living (ADL), heart rate variability, weight, gait speed, toilet usage. The communication module may be configured and operable to upload the recorded data to a database. Optionally, the Chronic Heart Failure (CHF) prediction engine comprises a neural network such as a network of sigmoid function neurons. Additionally or alternatively, the at least one wellbeing prediction engine may comprise a fall detection system.

Another aspect of the disclosure is to introduce a body volume monitor comprising: a radar unit comprising: at least one transmitter antenna connected to an oscillator and configured to transmit electromagnetic waves towards a target region, and at least one receiver antenna configured to receive electromagnetic waves reflected by a subject located within the target region and operable to generate raw data; and a processor unit configured to receive raw data from the radar unit and operable to generate a body model based upon the received data, and further operable to calculate a body volume index for the subject.

In still another aspect a gait speed monitor is introduced comprising: a radar unit comprising: at least one transmitter antenna connected to an oscillator and configured to transmit electromagnetic waves towards an extended target region, and at least one receiver antenna configured to receive electromagnetic waves reflected by a subject located within the extended target region and operable to generate raw data; and a memory unit configured and operable to store the image data; a processor unit comprising: a data filter configured to receive said raw data, and operable to process the raw data to remove data relating to reflections from static objects thereby generating filtered data; a tracker module configured to receive the filtered data from the data filter and operable to process the filtered data to identify moving targets and to track the location of the moving targets over time thereby generating target data; and a gait classification module configured to receive the target data from the tracker module and operable to process the target data by applying gait classification rules and further operable to calculate a gait speed of the subject; and a communication module configured and operable to communicate information to third parties. Optionally, the extended target region has a length of at least five meters.

Another aspect is to teach a method for assessing ongoing wellbeing of at least one subject, the method comprising: providing at least one subject monitoring station configured and operable to collect health indication parameters from the at least one subject; providing a parameter collection database for storing monitored health indication parameters for the at least one subject; providing at least one wellbeing prediction engine; the wellbeing prediction engine accessing the parameter collection database; and executing a wellbeing predictive function thereby generating at least one wellbeing index for the at least one subject.

Where appropriate, the step of providing at least one wellbeing prediction engine comprises providing a machine learning CHF risk model, the method further comprising: populating the parameter collection database with training data by: monitoring health indication parameters of test-subjects over time; storing monitored health indication parameters for each test-subject; recording CHF status of each test-subject; training the machine learning CHF risk model using the training data; monitoring health indication parameters of a patient; inputting the health indication parameters of the patient into the machine learning CHF risk model; the machine learning CHF risk model generating a CHF risk index for the patient.

Optionally, the step of providing at least one subject monitoring station may comprise at least one step selected from: providing a body volume monitor configured and operable to record the body volume of the subject; providing a gait speed monitor configured and operable to record gait speed of the subject; providing a remote vital signs monitor configured and operable to record breathing rate and heart rate of the subject; providing an activity monitor configured and operable to record events indicative of activity of daily living of the subject; providing a body temperature monitor configured and operable to record body temperature of the subject; providing a weight monitor configured and operable to record weight of the subject; and providing a blood pressure monitor configured and operable to record blood pressure of the subject.

Where appropriate, the health indication parameters are selected from the group consisting of: body volume, body mass, gait speed, breathing rate, heart rate, heart rate variability, activity of daily living, body temperature, blood pressure and combinations thereof. Variously, the step of providing a machine learning CHF risk model comprises providing a non-linear model, a neural network, a network regression model or a network of sigmoid function neurons.

### BRIEF DESCRIPTION OF THE FIGURES

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the various selected embodiments may be put into practice. In the accompanying drawings:

FIG. 1A is a schematic representation of a system for ongoing health monitoring by remote examination of patients using radar based telemedical monitoring device;

FIG. 1B illustrates a schematic representation of the telemedical monitoring device 104 with attached external units;

FIG. 2 is a schematic flowchart illustrating an exemplary method for remote examination of the patient;

FIG. 3A is a schematic representation of a system for assessing risk of chronic heart failure (CHF);

FIG. 3B is a schematic representation of a system for populating a parameter collection database for training a machine learning CHF risk model;

FIG. 4A is a flowchart indicating selected actions in a method for assessing risk of Chronic Heart Failure (CHF) according to an embodiment of the invention;

FIG. 4B schematically represents a training system for a machine learning CHF risk model;

FIG. 5A is a block diagram schematically representing selected components of a body volume monitor;

FIG. 5B is a schematic representation of a possible example of an ongoing health and activity monitor system;

FIG. 5C is a schematic block diagram indicating data flow within an ongoing health and activity monitor system;

FIG. 6A is a schematic flowchart illustrating an exemplary method for populating a database with time dependent energy profiles according to an aspect of the invention;

FIG. 6B is a schematic flowchart illustrating an exemplary method for anomaly detection and alert generation according to an aspect of the invention;

FIG. 7 shows a set of standard energy profiles for a target area;

FIG. 8 shows a set of time dependent energy profiles for target segments of a target area;

FIGS. 9A, 10A, and 11A illustrate KL Divergence values over all time windows in case of normal behaviour in exemplary embodiments of the invention; and

FIGS. 9B, 10B and 11B illustrate KL Divergence values over all time windows in case of actual falls in exemplary embodiments of the invention.

### DETAILED DESCRIPTION

Aspects of the present disclosure relate to systems and methods for monitoring health and wellbeing in an ongoing manner. In particular the disclosure relates to radar based a subject monitoring system operable to collect risk parameters in communication with a prediction engine.

Radar chips may be used to scan a monitored region such as an enclosed room. The data obtained by the scanning radar chip may be processed to identify targets within the monitored region. The identified targets may be tracked and profiled to indicate their state of health.

It is noted that monitoring both health indication parameters and activity of subjects may allow the early identification of various ailments. By way of example, the onset of chronic heart failure (CHF) may be indicated by a reduction of a subject's weight. Similarly, irregular breathing and heart rate may indicate a risk of sleep apnea. Increased frequency of toilet visits may go unnoticed by the subject but may indicate a high risk of urinary tract infection. Slower movements and changed gait while walking may indicate a higher risk of falling or the onset of dementia for example.

It is a particular aspect of the current disclosure to provide a passive monitor which may gather health indication parameters more passively in an ongoing manner so as to provide an indication of risk of ailments in at least one subject.

Accordingly, systems and methods are introduced for monitoring health and assessing risk of ailments. Various examples of passive monitors are described herein which may be combined to collect relevant health indication parameters and activity monitoring parameters. Such parameters may be communicated to a wellbeing prediction engine operable to generate wellebeing index for the monitored subject.

For illustrative purposes, it is noted that subjects at risk of chronic heart failure (CHF) are typically required to actively measure and report their weight, however they are often reluctant to do so. Consequently, the onset of CHF often goes undetected and preventative measures are not timely taken to prevent deterioration.

It has been found that apart from weight of a subject, other health parameters may be good predictors of CHF risk. Such risk parameters include but are not limited to body volume, body mass, gait speed, breathing rate, heart rate, heart rate variability, activity of daily living, body temperature, blood pressure and the like as well as combinations thereof.

Various passive monitors such as described herein may be combined to collect relevant risk parameters and to communicate these to a CHF prediction engine operable to process the multiple risk parameters and thereby to calculate a CHF risk index of the monitored subject.

Examples of CHF prediction engines include local processors operable to execute code stored upon memory units, the code directed to applying a CHF predictive function upon the input parameters. The CHF predictive function may be a locally stored program for calculating risk by combining the risks indicated by each risk parameter into a general characteristic risk value.

Additionally or alternatively, the CHF prediction engine may include a machine learning CHF risk model trained to output a risk from input data. It is particularly noted that the subject monitoring stations herein described may be used to harvest risk parameters from multiple subjects and to upload such data to a central parameter collection database which may be used to produce training data for such a machine learning CHF risk model.

It will be appreciated that similar wellbeing prediction engines may be developed to use these or other collected health and activity parameters to generate an index for other ailments as required, such as for heart attack risk, sleep apnea, urinary track infection, dementia, depression and the like as well as combinations thereof.

As required, detailed embodiments of the present invention are disclosed herein; however, it is to be understood that the disclosed embodiments are merely examples of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

As appropriate, in various embodiments of the disclosure, one or more tasks as described herein may be performed by a data processor, such as a computing platform or distributed computing system for executing a plurality of instructions. Optionally, the data processor includes or accesses a volatile memory for storing instructions, data or the like. Additionally or alternatively, the data processor may access a non-volatile storage, for example, a magnetic hard disk, flash-drive, removable media or the like, for storing instructions and/or data.

It is particularly noted that the systems and methods of the disclosure herein may not be limited in its application to the details of construction and the arrangement of the components or methods set forth in the description or illustrated in the drawings and examples. The systems and methods of the disclosure may be capable of other embodiments, or of being practiced and carried out in various ways and technologies.

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting. Accordingly, various embodiments may omit, substitute, or add various procedures or components as appropriate. For instance, the methods may be performed in an order different from described, and various steps may be added, omitted or combined. In addition, aspects and components described with respect to certain embodiments may be combined in various other embodiments.

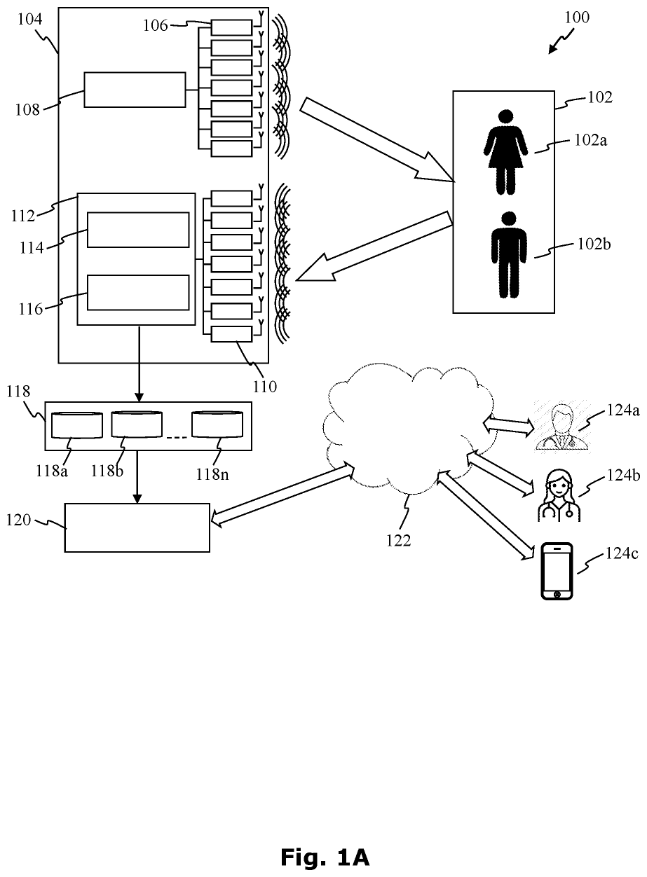

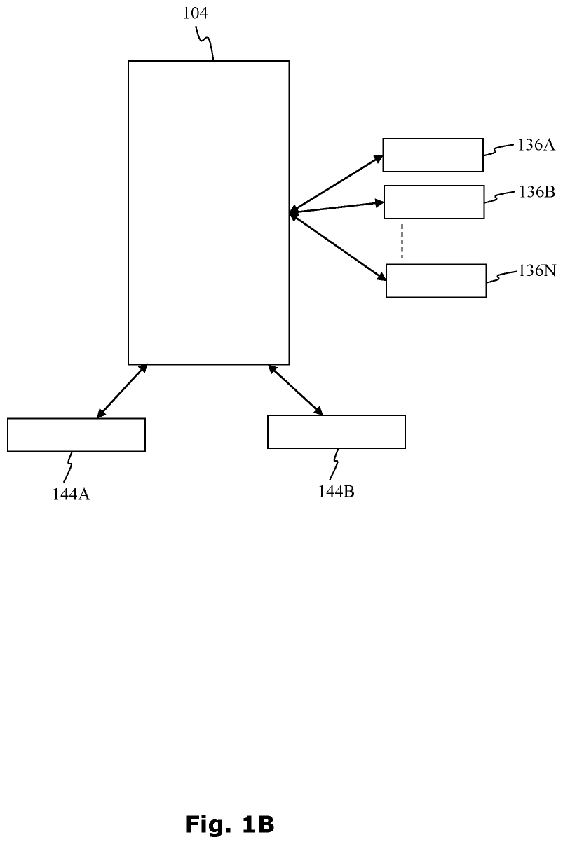

Reference is now made to FIG. 1A, which is a schematic representation of a system 100 for remote examination of patients. The system 100 includes a radar-based telemedical monitoring device 104, a database 118 and a communicator 120.

The radar-based telemedical monitoring device 104 includes an array of transmitters 106 and an array of receivers 110. The array of transmitters 106 may include an oscillator 108 connected to at least one transmitter antenna or an array of transmitter antennas 106. Accordingly, the transmitters 106 may be configured to produce a beam of electromagnetic radiations, such as microwave radiation or the like, directed towards a monitored region 102 such as an enclosed room, a particular arear of the hospital room, or the like. The receiver 110 may include an array of receiver antennas configured and operable to receive electromagnetic waves reflected by objects within the monitored region 102. The monitored region 102 is shown to include two patients 102A and 102B. However, monitored region 102 may include a smaller area focusing on one patient or a larger area focusing on a large number of patients for measuring the physical parameters without limiting the scope of the invention.

In a particular embodiment, the telemedical monitoring device 104 monitors the patients 102A and 102B without any physical contact or attachments. The telemedical monitoring device 104 may be appropriately positioned at a distance of a few feet from the monitored region 102 to effectively monitor the patients 102A and 102B. In one embodiment, the telemedical monitoring device 104 is positioned at the head/foot of a bed or proximate to a chair (not shown) on which the subject 102A is resting. The telemedical monitoring device 104 may also be positioned on a table or wall adjacent or opposite the bed (not shown), or on the ceiling of the room to monitor the patients 102A and 102B. In a room of a large number of patients, the telemedical monitoring device 104 may be placed at a center position to capture information from all the patients.

The information received by the receiver 110 of the telemedical monitoring device 104 includes various physical parameters of the patients 102A and 102B along with patients' profiles. The physical parameters which may be monitored by the telemedical monitoring device 104 include, but are not limited to, the heart rate, heart variability, respiratory rate, sleep scores, gait, postures, etc. The patient profile includes various information of the patient including, but not limited to, name, age, gender, residence address, profession, dietary information, medical history, current treatment, etc.

The electromagnetic signals received by the receiver 110 is sent to a processing unit 112 of the telemedical monitoring device 104. The processing unit 112 comprises a subject identifying unit 114 which filters out the non-desired signals received from other objects present in the monitored region 102, such as a table, chair, bed, etc. the process of filtering out the non-desired signals is beyond the scope of the present invention. The subject identifying unit 114 also distinctly identifies the signals received from different subject patients. For example, subject identifying unit 114 distinctly identifies the signals received from patients 102A and 102B and transfers the data to a data analyzing unit 116 for further processing. The data analyzing unit 116 analyzes the signals for various monitored parameters, including but not limited to, the heart rate, heart variability, respiratory rate, sleep scores, posture, gait, etc. The data analyzing unit 116 may prepare separate health profiles for the patients 102A and 102B including the monitored parameters. The data analyzing unit 116 may also prepare health reports for patients, including but not limited to, an inspection report, a palpation report, a percussion report, an auscultation report and a neurologic examination report.

The health profiles and health reports of patients are stored in the database 118. The health profiles and health reports 118a . . . 118n of each patient are stored individually in the database 118.

As and when required, the health profiles and health reports of individual patients are sent to the medical examiner for monitoring and treatment. The health profiles and health reports are sent from the database 118 through a communicator 120 which transmits the information to a medical examiner 124A through a communication network 122. The communication network 122 may include a Bluetooth network, a Wired LAN, a Wireless LAN, a WiFi Network, a Zigbee Network, a Z-Wave Network or an Ethernet Network. The health profiles and health reports may be sent to multiple doctors 124a, 124b, etc. who are involved in the treatment. The health profiles and health reports may also be sent to a communication device 124c of a caretaker of the patient.

FIG. 1B illustrates a schematic representation of the telemedical monitoring device 104 with attached external units. In a particular embodiment, the telemedical monitoring device 104 may connect to various other medical devices for measuring the patient's 102A and 102B parameters. The telemedical monitoring device 104 is shown here connected to a weight measuring unit 214A and a blood pressure monitoring unit 214B. The units 102A and 102B measure the weight and blood pressure of the patient 102A and 102B and transfer the data to the telemedical monitoring device 104. The telemedical monitoring device 104 may also connect to a number of sensors 136A . . . 136N, like, an acoustic sensor, an infrared body temperature sensor, and other sensors that measure parameters such as ambient humidity, temperature and light level. The integrated data may be used to assess a health condition of the patients 102A and 102B. The weight measuring unit 136A, the blood pressure monitoring unit 144B and sensors 136A . . . 136N may be connected to the telemedical monitoring device 104 via a Bluetooth connection, a Wired LAN connection, a Wireless LAN connection, a WiFi connection, a Zigbee connection, a Z-Wave connection or an Ethernet Network connection. The telemedical monitoring device 104 is disclosed here connected to two external measuring units, however, it may connect to any other medical device without limiting the scope of the invention. Exemplary medical devices include, but are not limited to, a pulse oximeter monitoring unit, etc.

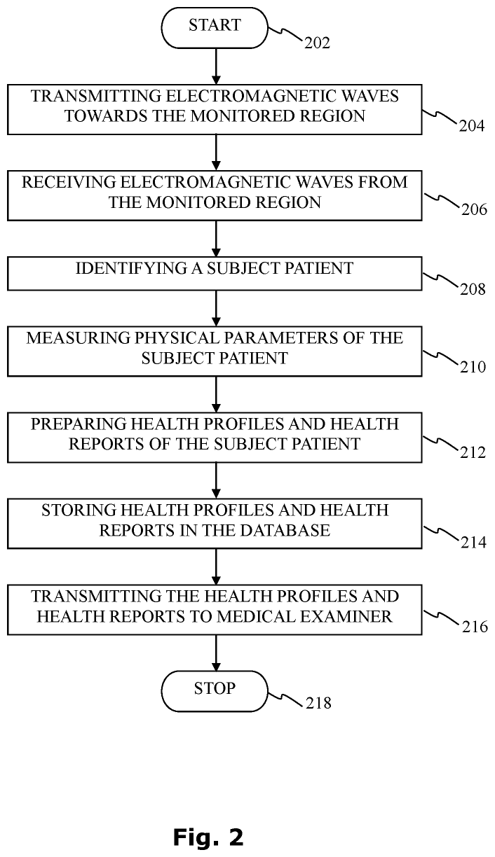

Referring to FIG. 2 which is a schematic flowchart illustrating an exemplary method for remote examination of the patient according to an aspect of the invention. The process starts at step 202 and electromagnetic waves (EM) are transmitted by the transmitter 106 of the telemedical monitoring device 104 towards the monitored region 102 at step 204. The EM waves reflected from the monitored region 102 are received by the receiver 110 at step 206. The received EM signals are transferred to the subject identifying unit 114 of the processing unit 112. At step 208, the subject identifying unit 114 filters out the non-desired data and identifies the data of the desired subject. As required, the subject identifying unit 114 may select the data of one subject patient, e.g. patient 102A or multiple subject patients, e.g. patients 102A and 102B. At step 210, the data analyzing unit 116 measures the physical parameters of the subject patient 102A and prepares health profiles and health records of the patient 102A at step 212. The health profiles and health records of the patient 102A are stored in the database 118 at step 214. As and when required, at step 216, the health profiles and health records of the patient 102A are sent to one or more of the medical practitioners 124A and 124B to assess the medical condition of the patient 102A and suggest appropriate treatment. The health profiles and health records of the patient 102A may also be sent to the communication device 124C of the caretaker of the patient 102A. The process is completed at step 218.

The systems and methods explained above may perform physical examination of the patient remotely and non-intrusively. The examination report of the patient may be sent to the doctor for treatment advice.

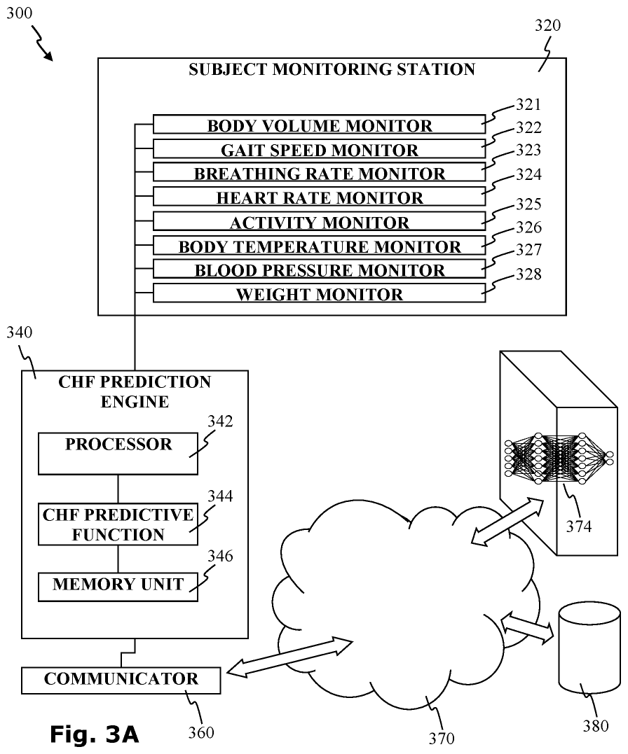

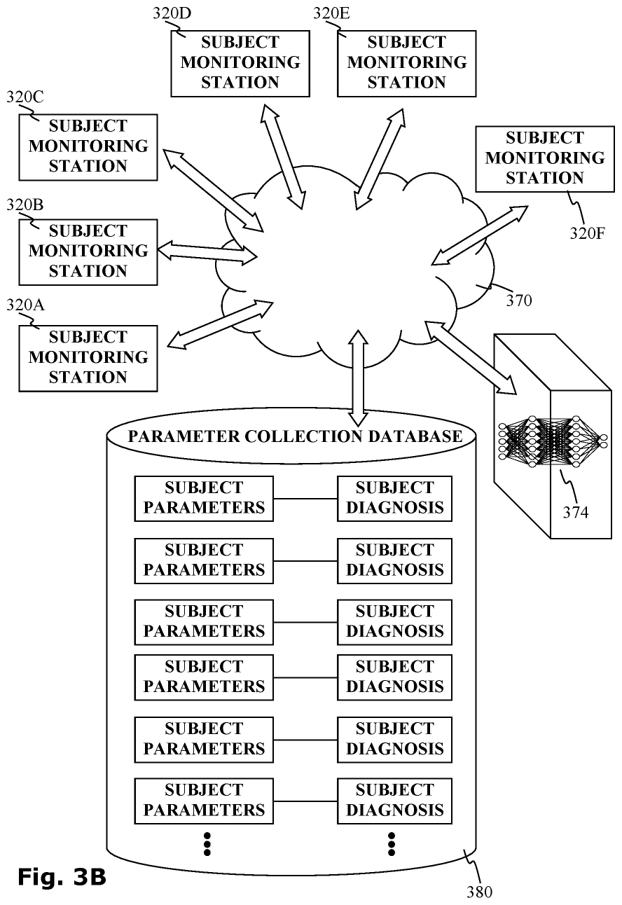

Reference is now made to FIG. 3A which shows a schematic representation of a system 300 for assessing risk of chronic heart failure (CHF). The system 300 includes a subject monitoring station 320, a chronic heart failure (CHF) prediction engine 340. The system further includes a communicator 360 for connecting the CHF prediction engine 340 with a computer network 370, such as the internet, including a remote parameter collection database 380 and a computerized CHF risk model 374.

The subject monitoring station 320, may include various parameter collectors such as a body volume monitor 321 configured and operable to record the body volume of the subject, a gait speed monitor 322 configured and operable to record gait speed of the subject, a breathing rate monitor 323, a breathing rate monitor 324 configured and operable to record the breathing rate of the subject, a heart rate monitor configured and operable to record the heart rate 324 of the subject, an activity monitor 325 configured and operable to record events indicative of activity of daily living of the subject, such as toilet usage, sleep time, food preparation and the like; a body temperature monitor 326 configured and operable to record the body temperature of the subject; a blood pressure monitor 327 configured and operable to record the blood pressure of the subject; and a weight monitor 328, such as a scale, for recording the weight of the subject.

It is a particular feature of the currently disclosed system for assessing risk of chronic heart failure (CHF) that where possible, passive monitors are used to collect the risk parameters. For example radar based monitors, such as described herein may be used to collect data pertaining to body volume, gait speed, breathing rate, heart rate and activity. Infrared thermometers systems may be used to measure body temperature, and underfloor scales may be used to measure weight for example.

A further feature of the system 300 is that the monitors may gather these parameters without infringing the privacy of the subject. It is therefore noted that radar based systems which do not rely upon image collection or indeed capture any images at all may be preferred to image capture devices such as video cameras.

The Chronic Heart Failure (CHF) prediction engine 340 may comprise a memory unit 346 and a processor 342. The memory unit 346 may be configured to store recorded data generated by the monitors. Accordingly, the processor 342 may be configured and operable to access the recorded data stored in the memory unit and to execute a Chronic Heart Failure (CHF) predictive function thereby generating a CHF risk index for the subject.

Where appropriate the communicator 360 may be included to connect the CHF prediction engine 340 with third parties or to the remote parameter collection database or computerized CHF risk model possibly via a computer network 370.

With reference now to FIG. 3B a system is presented for populating a parameter collection database 380 for training a machine learning CHF risk model 374. The system includes a plurality of subject monitoring stations 320A-F, a parameter collection database and a CHF risk model server connected via a computer network 370.

The subject monitoring stations 320A-F are configured to collect risk parameters relating to individual subjects and to communicate packages of risk parameter data to the parameter collection database. Preferably, risk parameter data packages may further include a subject diagnosis, possibly performed by a medical professional, which may updates over time if the subject later develops CHF. It is noted that the packages of risk parameter data may be anonymous so as to preserve compromising patient privacy.

The parameter collection database 380 may thereby be populated with multiple individual subject records. The parameter collection database 380 may be used to provide training data for a CHF risk model. A method for training the CHF risk model is described herein.

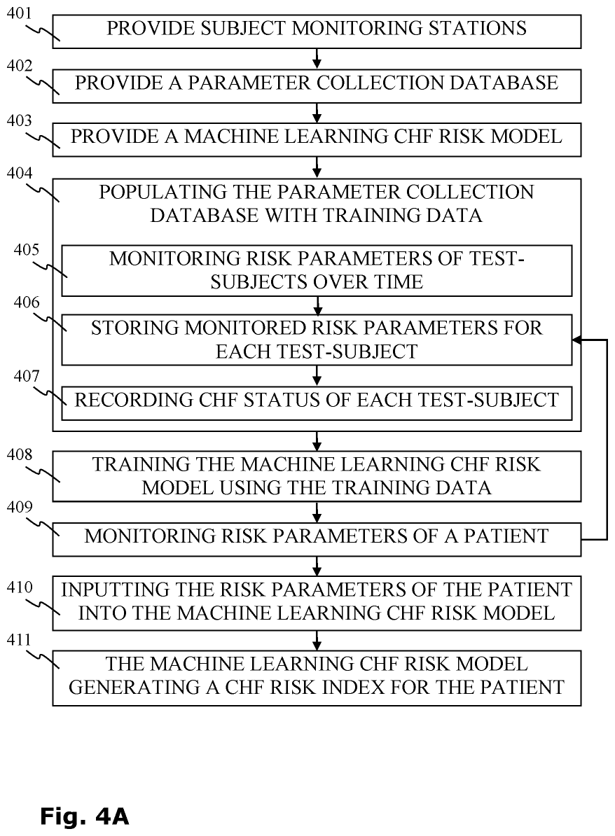

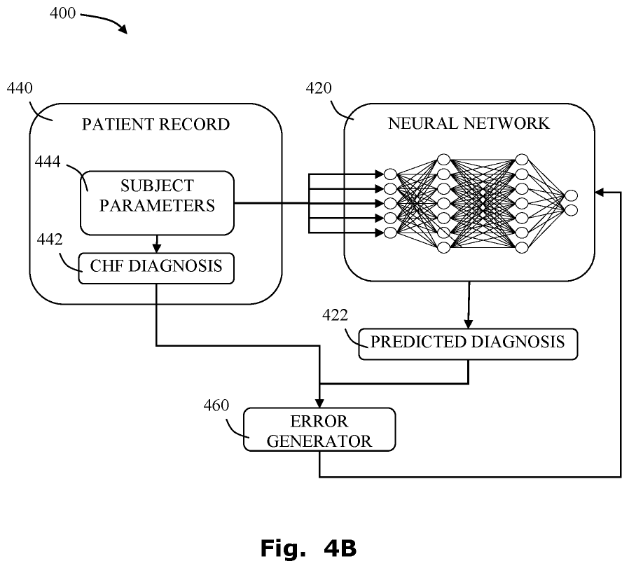

Reference is now made to the flowchart of FIG. 4A which presents selected actions in a method for assessing risk of Chronic Heart Failure (CHF). The method includes a training data collection phase, a training phase and a monitoring phase.

Data is collected by providing subject monitoring stations 401 configured and operable to collect risk parameters from subjects, such as body volume, body mass, gait speed, breathing rate, heart rate, heart rate variability, activity of daily living, body temperature, blood pressure and the like as well as combinations thereof.

The method further includes providing a parameter collection database 402 for storing monitored risk parameters for each subject; providing a machine learning CHF risk model 403; and populating the parameter collection database 404 with training data by: monitoring risk parameters of test-subjects over time 405; storing monitored risk parameters for each test-subject 406; and recording CHF status of each test-subject 407, such as by recording the diagnosis of a medical professional.

Having populated the training database, the method continues with training the machine learning CHF risk model using the training data 408.

In the monitoring phase, the CHF risk model is used by monitoring risk parameters of a patient 409; inputting the risk parameters of the patient into the machine learning CHF risk model 410; and the CHF risk model generating a CHF risk index for the patient 411. It is further noted that during the monitoring phase, newly collected data may be further stored in the collection database so as to improve the training of the CHF in an ongoing manner.

Reference is now made to the block diagram of FIG. 4B, representing the main components of a possible training system 400 for generating a CHF risk model using supervised learning. Such a training system 400 is presented by way of illustration and may be used during set up.

Various models maybe used such as neural networks, non-linear models, network regression models, networks of sigmoid function neurons and the like. For the purposes of illustration a neural network is described herein in however, other models and training systems will occur to those skilled in the art.

The training system 400, of the example includes a neural network 420 a real patient record 440 and an error generator 460. The real patient record includes some real CHF diagnosis associated with the patient output 442 and the neural network generates a predicted output 422. The Error generator 860 compares the actual output signal 442 and the predicted output 422 producing a cost function which is fed back to the neural network which optimizes the various neuron parameters so as to minimize the cost function, possibly using iterative techniques or heuristic techniques.

By way of example a cost function may be generated by a controller summing the squares of the errors for each input, although other cost functions may be preferred as suit requirements.

Having generated a cost function, the controller may adjust the neuron parameters so as to minimize the cost function. Minimization algorithms may include, but are not limited to heuristic methods such as Memetic algorithms, Differential evolution, Evolutionary algorithms, Dynamic relaxation, Genetic algorithms, Hill climbing with random restart, Nelder-Mead simplicial heuristic: A popular heuristic for approximate minimization (without calling gradients), Particle swarm optimization, Gravitational search algorithm, Artificial bee colony optimization, Simulated annealing, Stochastic tunneling, Tabu search, Reactive Search Optimization (RSO) or the like. Additionally or alternatively, minimization may include iterative methods such as Newton's method, Sequential quadratic programming, Interior point methods, Coordinate descent methods, Conjugate gradient methods, Gradient descent, Subgradient methods, Bundle method of descent, Ellipsoid methods, Reduced gradient method, Quasi-Newton methods, Simultaneous perturbation stochastic approximation (SPSA) method for stochastic optimization, Interpolation methods and the like.

It is a particular feature of the training system 400 that the real patient record provides subject parameters 444 to the neural network, such that the neural network is optimized to produce a predicted diagnosis 422 as close as possible to the CHF diagnosis 442 of the real patient record for any given set of subject parameters.

Accordingly, once trained the neural network 420 is able to mimic a real patient, generating a predicted diagnosis 422 according to the monitored parameters such as body volume, body mass, gait speed, breathing rate, heart rate, heart rate variability, activity of daily living, body temperature, blood pressure and the like which may be provided as inputs.

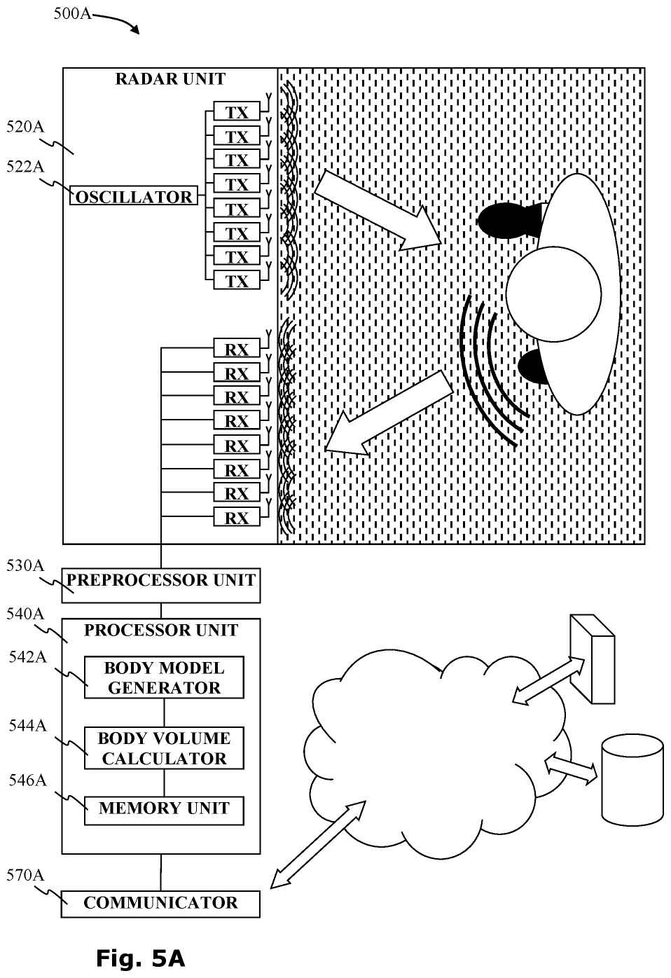

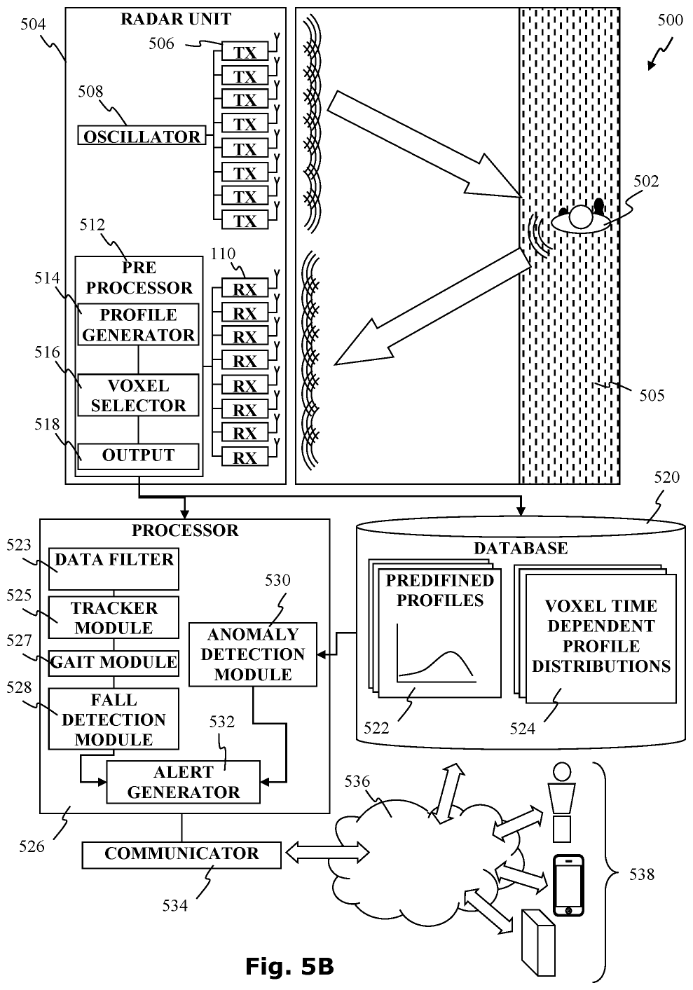

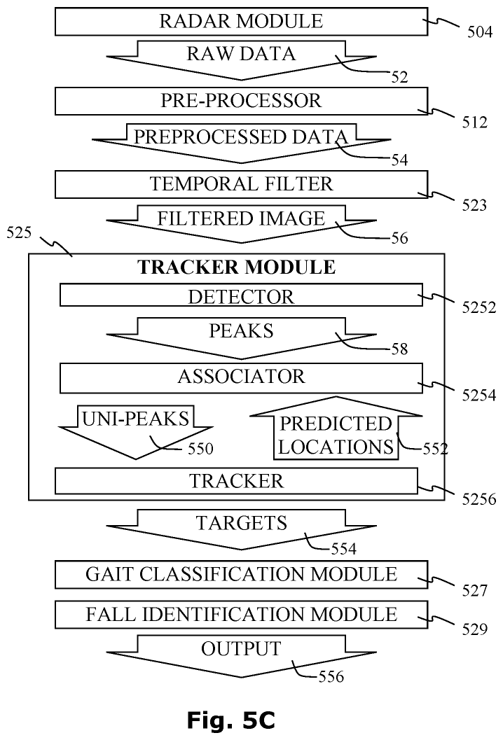

Referring now to FIG. 5A a possible example of a radar based body volume monitor 500A for use in a subject monitoring station is which schematically represented.

The body volume monitor 500A is operable to generate a value for a body volume index of a subject standing in a target zone. The body volume monitor may include a radar unit 520A directed towards the target zone and a processor unit 540A.

The radar unit may be mounted to a wall for example behind an optical mirror transparent to radiowaves, embedded in the frame of a mirror, or the like where it may scan a target region in front of the wall. The radar typically includes at least one array of radio frequency transmitter antennas and at least one array of radio frequency receiver antennas. The radio frequency transmitter antennas TX are connected to an oscillator 522A (radio frequency signal source) and are configured and operable to transmit electromagnetic waves towards the target region. The radio frequency receiver antennas RX are configured to receive electromagnetic waves reflected back from objects within the target region.

Such scanning arrangements are described further in the applicant's co-pending United States international patent application serial number PCT/IB2020/062121 which is hereby incorporated by reference herein in its entirety. The arrangement may be embedded in a wall, a mirror frame, a window, under the floor, in a ceiling, behind an optical mirror transparent to radio waves or the like as required.

Additionally or alternatively the scanning arrangement itself be directed towards a mirror surface and may be configured and operable to extend the target region into the virtual reflected region inside the mirror. Accordingly, shielded or eclipsed regions of the subject may be rendered visible by reflection within the mirror.

The raw data generated by the receivers is typically a set of magnitude and phase measurements corresponding to the waves scattered back from the objects in front of the array. Spatial reconstruction processing may be applied to the measurements to reconstruct the amplitude (scattering strength) at the three dimensional coordinates of interest within the target region. Thus each three dimensional section of the volume within the target region may represented by a voxel defined by four values corresponding to an x-coordinate, a y-coordinate, a z-coordinate, and an amplitude value.

Typically the receivers may be connected to a pre-processing unit 530A configured and operable to process the amplitude matrix of raw data generated by the receivers and produce a filtered point cloud suitable for model optimization.

Accordingly, where appropriate, a preprocessing unit 530A may include an amplitude filter operable to select voxels having amplitude above a required threshold and a voxel selector operable to reduce the number of voxels in the filtered data, for example by sampling the data or clustering neighboring voxels. In this manner the filtered point cloud may be output to a processor. It is further note that the filtered point cloud may further be simplified by setting the amplitude value of each voxel to ONE when the amplitude is above the threshold and to ZERO when the amplitude is below the threshold.

The processor 540A which is in communication with the preprocessor unit 530A may include a body model generator 542A operable to receive the filtered point cloud from the output of the preprocessor and to compare the filtered point cloud with a human parametric model stored in a memory unit 546A to generate a body model.

The parametric model may be generated by averaging scans of multiple subjects and/or applying machine learning to such scans and stored in the memory unit of the processor or remotely. The parametric model may be represented as a model function which receives a set of values representing model parameters and returns as set of voxels which model the subject.

By way of example, parameters may be selected from various measurable values of a subject, for example for a human subject parameters such as gender, height, weight, waist size, inner-thigh, inseam, arm-span, hand span, wrist to shoulder length, shoe size and the like as well as combinations thereof may generate candidate models with characteristic voxel sets. In some examples, separate parametric models may be provided for male and female subjects.

Accordingly, the processor may further include an optimizer and a parameter selector. The optimizer may further be configured and operable to compare the positions of each voxel in the parametric model with each voxel in the filtered point cloud. The parameter selector may be operable to receive the results of the comparison and to adjust the parameters accordingly so as generate a new candidate model. Once the optimizer reaches an optimal model wherein no further adjustment significantly improves the candidate model, that candidate model may be selected as the best fit model of the scanned subject. The subject may itself be characterized by the measurements used as parameter values for generating the best fit body model.

The processor may further include a body volume calculator 544A operable to analyze the body model of the subject in order to calculate a characteristic value for the body volume index of the subject.

Reference is now made to FIG. 5B which is a schematic representation of a possible heath monitoring system 500. The heath monitoring system 500 includes a radar unit 504, a processor unit 526 and a communication module 534.

The radar unit 504 includes an array of transmitters 506 and receivers 510. The transmitter may include an oscillator 508 connected to at least one transmitter antenna TX or an array of transmitter antennas. 506 Accordingly the transmitter may be configured to produce a beam of electromagnetic radiation, such as microwave radiation or the like, directed towards a monitored region 505 such as an enclosed room or the like. The receiver may include at least one receiving antenna RX or an array of receiver antennas 510 configured and operable to receive electromagnetic waves reflected by objects 502 within the monitored region 505.

The processor unit, 526 which may include modules such as a data filter 523, a tracker module 525, a gait classification module 527 and a fall identification module 529, may be configured to receive data from the radar unit 504 and be operable to generate fall alerts based upon the received data. Where appropriate, a preprocessor 512 may be provided to process the raw data before transferring the data to the processor unit 526, as described herein.

The communication module 534 is configured and operable to communicate with to third parties 538. Optionally the communication module 534 may be in communication with a computer network 536 such as the internet via which it may communicate alerts to third parties 538 for example via telephones, computers, wearable devices or the like.

It is noted that the system may further include a radar based passive gait speed monitor 527 for use in the subject monitoring station which is schematically represented. The gait speed monitor 527 may be operable to generate a value for the gait speed of a subject passing along an extended target zone 505. The gait speed monitor includes at least one radar scanning arrangement and a processor unit.

The radar scanning arrangement 504 is configured to monitor the movement of a subject 502 over an extended range. The extended range 505 is of dimensions suitable for the measurement of speed of sustained gait along a path of say 4-8 meters. Thus, by way of example, it may be preferred to locate a scanning arrangement to cover movement in a target zone of say 5-6 meters squared.

Where appropriate a single radar scanning arrangement may be used to monitor the entire length of the extended target zone, however where required multiple scanning arrangements may be preferred. The radar typically includes at least one array of radio frequency transmitter antennas and at least one array of radio frequency receiver antennas. The radio frequency transmitter antennas are connected to an oscillator (radio frequency signal source) and are configured and operable to transmit electromagnetic waves towards the target region. The radio frequency receiver antennas are configured to receive electromagnetic waves reflected back from objects within the target region.

The processor unit 526, which may include modules such as a data filter 523, a tracker module 525 and a gait classification module 527, may therefore be configured to receive data from the radar unit and be operable to process the target data by applying gait classification rules and further operable to calculate a gait speed of the subject.

Reference is now made to the block diagram of FIG. 5B indicating possible data flow through the health monitoring system 500. Raw data is generated by the radar module 504 which typically includes amplitude values for energy reflected at specific angles and ranges. The raw data 52 may be represented as images in polar coordinates. The preprocessor unit 512 may receive the raw data 52 from the radar module 504. The preprocessor unit 512 include a profile generator 514, a voxel selector 516 and an output 518.

The data filter 523 receives the raw data 52 directly from the radar module 504 or alternatively may receive pre-processed data 54 from the preprocessor unit 512. The data filter 523 may include a temporal filter operable to process the raw data 52 in order to remove all data relating to reflections from static objects. The filter 523 may thereby generate a filtered image 56 which includes only data pertaining to moving objects within the monitored region with background removed.

In certain examples, the data filter 523 may include a memory unit, and a microprocessor. Accordingly, the data filter 523 may store in the memory unit both a first set of raw data set from a first frame and a second set of raw data set from a second frame following a time interval. The microprocessor may be operable to subtract the first frame data from the second fame data thereby generating the filtered frame data. Other methods for filtering data will occur to those skilled in the art.

The filtered image data 56 may be transferred to a tracker module 525 operable to process the filtered image data 56 in order to identify moving targets with the data and to track the location of the identified moving targets over time thereby generating target data 554.

The tracker module 525 may include a detector 5252, an associator 5254 and a tracker 5256 and is operable to generate data 554 relating to targets within the monitored region. The detector 5252 receives the filtered image data 556 from the temporal filter 523 and processes the filtered image data 56 to detect local maxima peaks 558 within its energy distribution.

The peaks data 58 may be transferred to the associator 5254. The associator 5254 is operable to store the peak data 58 for each frame in a memory element and to associate each peak with a target object and further generating a single peak location (uni-peak) for each target.

The tracker 525 may be configured to receive target data from each frame and be operable to populate a target database with a location value and a speed value for each target in each frame, thereby generating tracking data which may be used to calculate predicted locations 552 for each target in each frame. By way of example,

The associator 5254 may be further operable to receive tracking data from a target tracker 5256. Accordingly when a uni-peak 550 coincides with the expected location of an existing target the peak may be associated with that existing target. Alternatively, where the location of the peak does not coincide with any tracked target the peak may be associated with a new target.

Target data 554 may be transferred to a gait classification module 527 and/or a fall identification module 529 operable to process the target data 554 by applying fall detection rules and to generate fall alert outputs 556 where required.

According to some examples, the fall identification module 529 includes a posture detector and a fall detector. The posture detector may be configured to store target data in a memory unit, to generate an energy profile for each target, and to apply posture selection rules thereby selecting a posture for each target. The posture detector may be further operable to store a posture history for each target in the memory unit. The fall detector may then access the posture history from the memory unit and generate a fall alert if at least one target is identified as fallen.

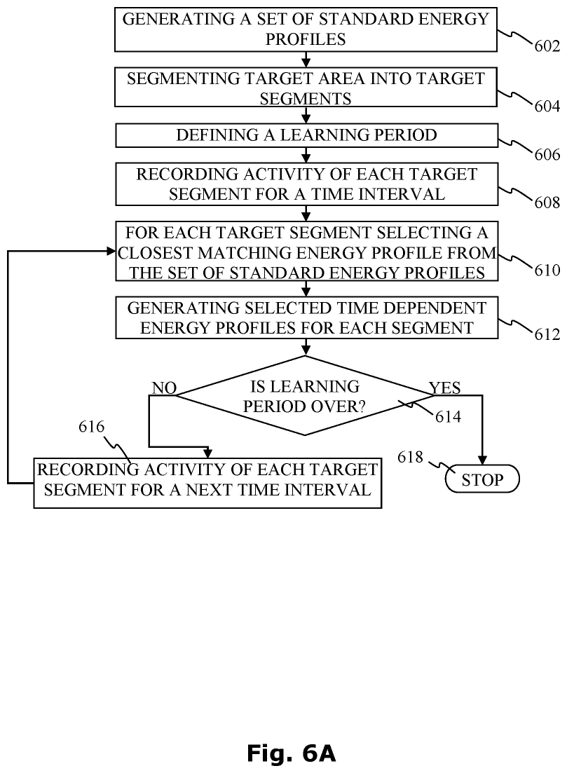

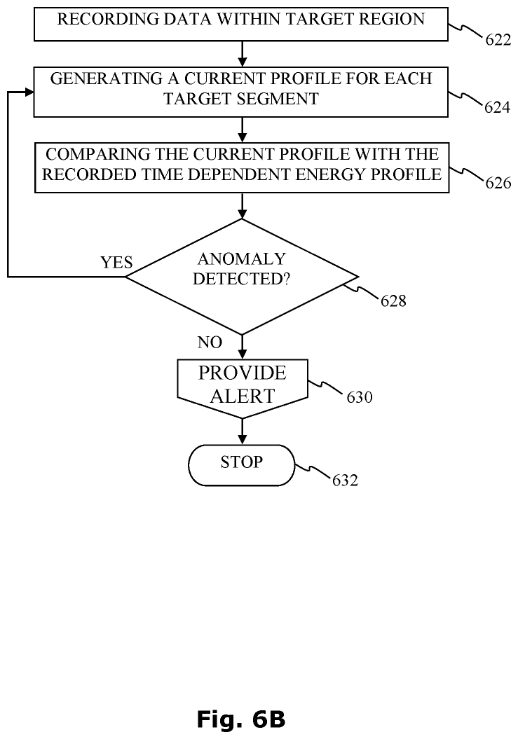

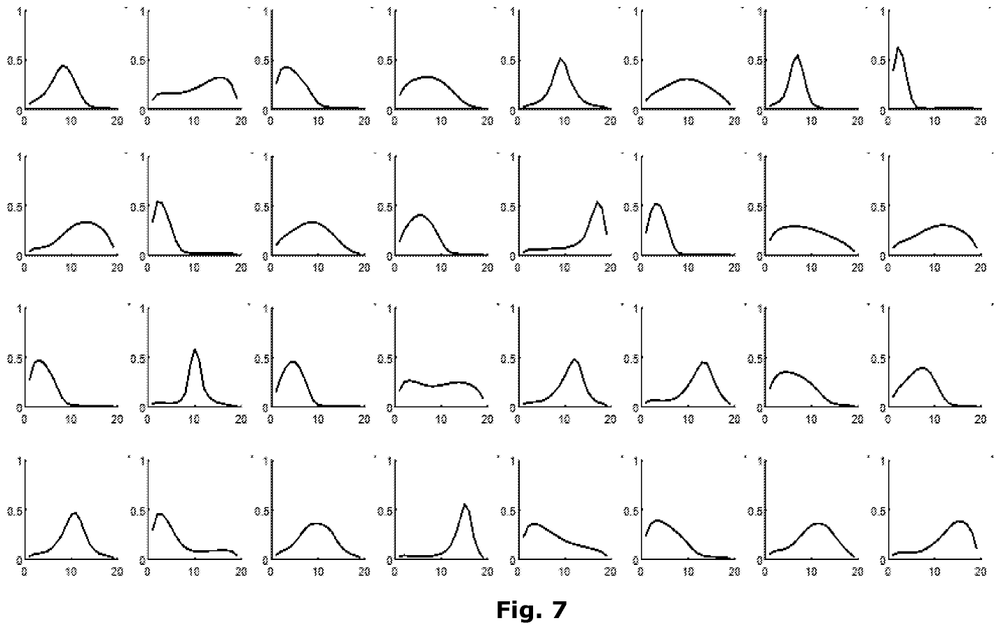

Referring to FIG. 6A which illustrates an exemplary method for populating a database with time dependent energy profiles. The time dependent energy profile for each section of the target area shows the relative likelihood of each of the set of energy profile being selected at a given time of day. The process starts at step 602 at which a set of standard energy profiles are generated and stored in the database. The set of standard energy profiles characterize the expected energy distribution associated with a subject in a different pose (standing, sitting, lying, walking, bending over etc. . . . ). A set of 32 standard energy profiles of an exemplary subject are shown in FIG. 7. These standard energy profiles are generated from large sample of data collected over a large period of time.

At step 604, the target area is segmented into a number of target segments by the segment selector. A learning period for collecting time dependent data is defined at step 606. In an exemplary embodiment, a learning period of 48 hours is defined with time intervals of 1 hour. At step 608, for each time interval, activity of each target segment is recorded. The activity is recorded through the reflections received from the target segments by the receiver of the radar unit. At step 610, the profile generator selects a closest match for the target segment from the set of standard energy profiles and generates time dependent energy profiles 524 for each segment at step 612. The time dependent energy profiles 524 are stored in the database 520.

At step 614, it is determined if all time intervals of the learning period have been completed. It is noted that the system may continue gathering profiles in an ongoing manner during operation even after the learning period is over. Where required older data may be overwritten or purged. In this manner the previous 48 hours may always be divided into a number of time intervals, such as 24 or twelve time intervals as required.

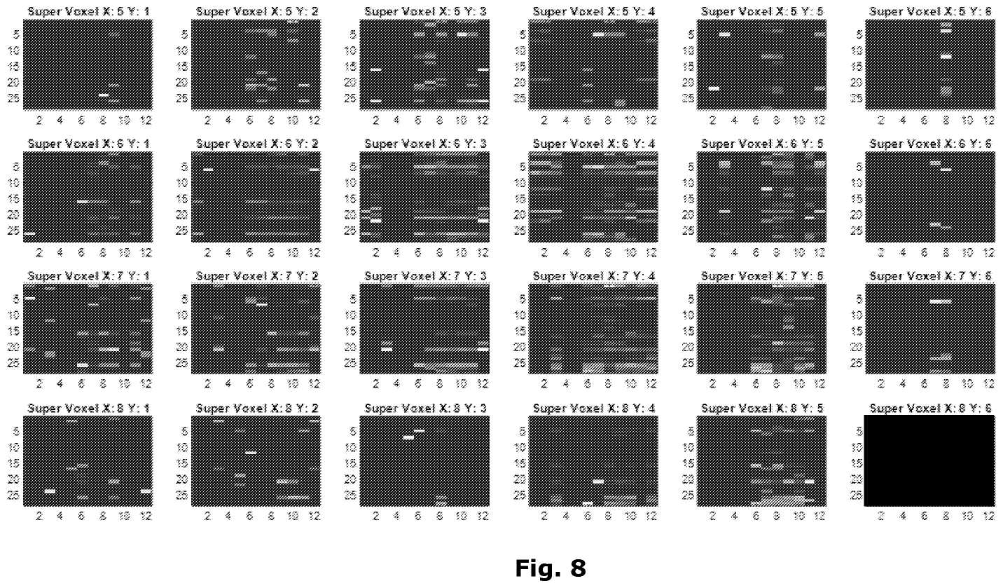

If “yes”, all time intervals of the learning period have been completed, then the process of populating the database 520 with time dependent energy profiles is completed and the process stops at step 618. Else, the activity of each target segment is recorded for the next time interval at step 616 and process repeats from step 610. FIG. 8 shows an exemplary set of time dependent energy profiles 524 for various target segments of a target area. The term “Super Voxel” herein refers to a “target segment” of the target area with ‘X’ and ‘Y’ coordinates defining the particular target segment.

Reference is now made to FIG. 6B which is a schematic flowchart illustrating an exemplary method for anomaly detection in fall alerts and alert generation. In case a fall is detected in the target region 502 based on the fall detection rules, at step 622, data corresponding to target region 502 is recorded by the receiver 110 of the radar unit 504. For each target segment of the target area 102, a current energy profile is generated by the profile generator 514 and sent to the processing unit 526 by the output unit 518 at step 624. At step 626, the current energy profile is compared with the recorded time dependent energy profile 524 stored in the database 520. Based on the comparison, it is determined if an anomaly is detected in the fall detection at step 628. In case no anomaly is detected in the fall detection, an alert is generated and provided to the intended recipients through various means at step 630. In case an anomaly is detected in the fall detection, the fall alert if filtered out and process repeats from step 624. The process completes at step 632.

In an exemplary embodiment, the process of anomaly detection in fall alerts is explained using Kullback-Leibler (KL) Divergence which measures how a probability distribution differs from a reference probability distribution. A metric Mⁱ is defined by the KL Divergence as:

$${M}^{i} ( {P}_{D}^{i} ‖ {P}_{W} ) = {\sum }_{v} {P}_{D}^{i} log ( \frac{{P}_{D}^{i}}{{P}_{W}} )$$

where, P_{W}ⁱ refers to time dependent energy profile distribution of a target segment; and P_{D} refers to the current energy profile distribution of the target segment.

A threshold T is defined such that if Mⁱ<T there is no anomaly in the fall detection. Consequently, a fall alert is generated and sent to the intended recipients. Otherwise, if Mⁱ≥T an anomaly is detected in the fall detection the fall detection is filtered out and no alert is generated.

Additionally or alternatively, an anomaly score may also be provided according to the confidence score based on the quality of information in the database and its diversity. A filter mechanism may be provided to perform a decision function base upon parameters such as the anomaly score and the like to further select appropriate alert generation.

It should be clearly understood that the process of anomaly detection in fall alerts explained using Kullback-Leibler (KL) Divergence is exemplary in nature and should not limit the scope of the invention. Any other suitable probability distribution function can be used for the purpose without limiting the scope of the invention.

FIGS. 16A, 17A and 18A illustrate KL Divergence values over all time windows in case of normal behavior in exemplary embodiments of the invention.

FIGS. 16B, 17B and 18B illustrate KL Divergence values over all time windows in case of actual falls in exemplary embodiments of the invention.

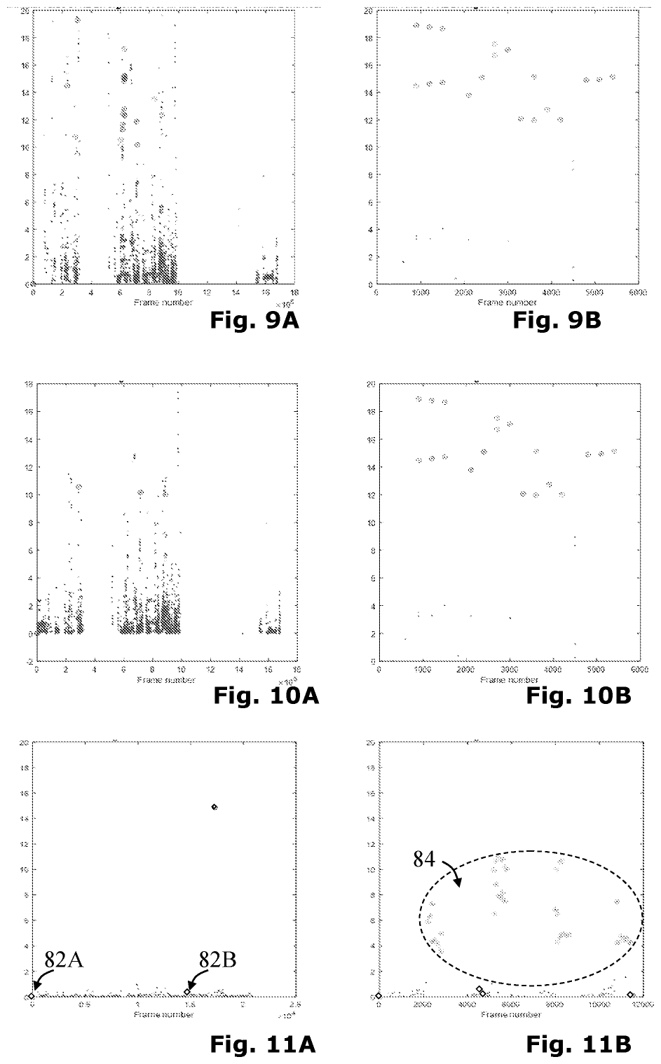

It is noted that the circled points in FIGS. 9A and 10A represent anomalies detected which do not correspond to actual falls. Such anomalies would not typically result in an alert being generated as they would not be accompanied by a fall detection event.

It is noted that the circled points in FIGS. 9B and 10B represent anomalies detected which correspond to actual falls. Such anomalies would typically be accompanied by a fall detection event and would therefore generate a fall alert.

FIGS. 9A and 10B represent divergence values recorded before the learning period was completed. By contrast, FIGS. 10A and 10B represent divergence values recorded after a learning period has been completed. Consequently more events are recorded as anomalous in FIG. 9A than in 10A although both these represent normal behavior.

Referring now to FIG. 11A, which shows KL divergence where no actual falls occur, it will be noted that although a number of fall detection events are recorded, as are circled in green, no corresponding anomaly was detected. Thus false positives are avoided.

By contrast, in FIG. 11B, where actual falls do occur, these generated fall detection events and are circled in green, it is noted that the events also correspond to anomalies. Accordingly, the fall detection alert is generated.

The systems and methods explained above provide an improvement to fall detection methodology by avoiding false positives.

Further features of the system include the capability to retain a long term memory for rare events, such as the operation of a washing machine or the like, which may otherwise be considered anomalies if only a 48 hour slice of memory is considered.

It is further noted that the system may classify zones within the target regions based upon the time dependent profiles. For example a zone may be identified to be a bed, if, say, a lying posture is detected over a long time mainly during night hours, or a toilet if, say, sitting and/or standing profiles are detected for characteristic short periods and so on. Such a classification system may form a basis for advanced room learning.

It is appreciated that certain features of the invention, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the invention has been described in conjunction with specific embodiments thereof, it is evident that other alternatives, modifications, variations and equivalents will be apparent to those skilled in the art. Accordingly, it is intended to embrace all such alternatives, modifications, variations and equivalents that fall within the spirit of the invention and the broad scope of the appended claims. Additionally, the various embodiments set forth hereinabove are described in terms of exemplary block diagrams, flow charts and other illustrations. As will be apparent to those of ordinary skill in the art, the illustrated embodiments and their various alternatives may be implemented without confinement to the illustrated examples. For example, a block diagram and the accompanying description should not be construed as mandating a particular architecture, layout or configuration.

### Technical Notes

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. It should be understood, therefore, that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

The scope of the disclosed subject matter is defined by the appended claims and includes both combinations and sub combinations of the various features described herein above as well as variations and modifications thereof, which would occur to persons skilled in the art upon reading the foregoing description.

1-9. (canceled)

10. A system for monitoring the ongoing wellbeing of at least one subject comprising:
at least one subject monitoring station configured and operable to collect health indication parameters from the at least one subject, the subject monitoring station comprising:
at least one radar unit comprising at least one transmitter antenna connected to an oscillator and configured to transmit electromagnetic waves towards a target region, and at least one receiver antenna configured to receive electromagnetic waves reflected by a subject located within the target region and operable to generate raw data; and
at least one processor configured to receive raw data from the radar unit and operable to generate said health indication parameters;
an activity monitor configured and operable to record events indicative of activity of daily living of the at least one subject;
a memory unit configured to store recorded data generated by the subject monitoring station and the activity monitor;
at least one wellbeing prediction engine comprising a processor configured and operable to access the recorded data stored in the memory unit and to execute a wellbeing predictive function thereby generating at least one wellbeing index for the at least one subject;
wherein the subject monitoring station comprises a gait speed monitor and wherein the processor further comprises:
a data filter configured to receive said raw data, and operable to process the raw data to remove data relating to reflections from static objects thereby generating filtered data;
a tracker module configured to receive the filtered data from the data filter and operable to process the filtered data to identify moving targets and to track the location of the moving targets over time thereby generating target data; and
a gait classification module configured to receive the target data from the tracker module and operable to process the target data by applying gait classification rules and further operable to calculate a gait speed of the subject.

11-17. (canceled)

18. A gait speed monitor comprising:
a radar unit comprising:
at least one transmitter antenna connected to an oscillator and configured to transmit electromagnetic waves towards an extended target region, and
at least one receiver antenna configured to receive electromagnetic waves reflected by a subject located within the extended target region and operable to generate raw data; and
a memory unit configured and operable to store the image data;
a processor unit comprising:
a data filter configured to receive said raw data, and operable to process the raw data to remove data relating to reflections from static objects thereby generating filtered data;
a tracker module configured to receive the filtered data from the data filter and operable to process the filtered data to identify moving targets and to track the location of the moving targets over time thereby generating target data; and
a gait classification module configured to receive the target data from the tracker module and operable to process the target data by applying gait classification rules and further operable to calculate a gait speed of the subject; and
a communication module configured and operable to communicate information to third parties.

19. The monitor of claim 18 wherein the extended target region has a length of at least five meters.

20-27. (canceled)
